(19) United States
(12) Patent Application Publication
(10) Pub. No.: US 2023/0142817 A1
(43) Pub. Date: May 11, 2023

(54) Increased isoelectric points for preventive compounds for covid 19 treatments

(71) Applicant: William M. Selenke, Cincinnati, OH (US)

(72) Inventor: William M. Selenke, Cincinnati, OH (US)

(21) Appl. No.: 17/300,780

(22) Filed: Nov. 5, 2021

(51) Int. Cl. A61K 31/714; A61K 31/525; A61K 33/30; A61K 31/506

(52) U.S. Cl. CPC A61K 31/714; A61K 31/525; A61K 33/30; A61K 31/506

(57) ABSTRACT

Covid 19 is a serious virus produces pandemic with great lethality. The present invention teaches that if the Covid 19 virus cannot enter the lung or other body cells that infection of Covid 19 can be reduced. The inventive element of the present invention is to saturate the megalin/cubilin receptor with cobalamin (vitamin B 12) that are specific for Covid 19 virus. This treatment using cobalamin may include additional compounds with increased isoelectric points.

### FIELD OF INVENTION

The field of the invention is Covid 19 Treatments. The seriousness of this disease has produced many investigations into its prevention and treatment. Prevention and treatment of Covid 19 consists primarily of four processes. The first it to prevent the person to person spread. This involves masks, social distancing, avoiding crowds, and hand washing. The second is to prevent the viral entrance into cell when the patient acquires the virus. The third is to prevent the replication of the virus within the cell. The fourth is to treat the cellular damage caused by the virus. The present invention is concerned with the prevention of the Covid 19 virus (and likely other viruses) entrance into the cell. The compounds of the present invention are administrated during the onset of Covid 19.

### BACKGROUND OF INVENTION

Because of the great numbers of afflicted patients worldwide, there were a number of published indications, on the Internet, of prevention/treatments of various agents discovered by case studies. A non-exhaustive list of these preventions/treatments include nicotine, [1] (an ingredient of tobacco with minimal medical uses) chloroquine [2] (a compound used primarily for the treatment of malaria), metformin [3] (widely used treatment for type II diabetes, [4] Famotidine, (Pepsid®, an over the counter remedy for heartburn) and zinc [5] (a necessary mineral for health). Cimetidine, [Recent investigations from the literature indicate that cimetidine an H2 blocker), Nitazoxanide (a broad spectrum antiparasitic and broad spectrum antiviral {Is its antiviral effects do to blocking megalin/cubilin?}) and Losartan (an anti-hypertension drug , which incidentally works by blocking angiotensin II {perhaps also blocking angiotensin II attachment to megalin/cubilin?}) Investigating these treatments to determine common characteristics may produce useful treatments. A hint that suggests treatments for Covid 19 is that these varied agents have a common characteristic, that is a high isoelectric point (pl).

Note, these compounds became pharmaceuticals because of the therapeutic effects. The purpose of this patent application is to determine compounds with high isoelectric points that have minimal physiological effects. The inventive element of the present invention is cobalamin administrated as inhalation mist.

The importance of increased isoelectric points for preventive agents is that a number of reports demonstrate that Covid 19 enters the cell by binding to angiotensin II. [6] The inventive element of the present invention is that it is that angiotensin II must bind to megalin/cubilin to be up taken by the cell [7]. As noted, the commonality of drugs mentioned in the above paragraph is their high isoelectric points (pl). Agents with a high isoelectric point binds directly with megalin/cubilin. If the negatively charged sites on megalin/cubilin are saturated with positively charged agents the Covid 19 angiotensin II complex cannot enter the cell because the required negative binding sites are saturated. Treatments agent for Covid 19 ideally should have with minimal physiological/pharmacological actions.

That is compounds with high isoelectric point, i.e., with isoelectric points similar with the angiotensin ii Covid 19 complex, such compounds should inhibit cellular uptake of the angiotensin II-Covid 19 complex. Thiamin, amprolium (a veterinary compound of almost no toxicity) metformin and vitamin B12 would be the starting test compounds. It is to be noted that these compounds must be given at very high doses, and perhaps as an aerosol inhalation mist. Both thiamin and amprolium should allow few physiological effects because both are readily removed from the circulation by the kidney. Excessive doses of these two compounds are removed by the kidney. A special note must be made for Vitamin B 12: Treatment with high dose vitamin B12 been shown to be safe for more than 50 years [7]

The present invention does teach only these few preventive agents (zinc, thiamin, amprolium and cobalamin [B12] as all-inclusive preventative agents. They are claimed because of their well-known minimal toxic effects. Other agents with isoelectric points similar to the angiotensin II-Covid 19 complex could be more useful. Preventive agents of the present invention should be administered either orally, parentally, or by inhalation mist.

### References

1] Makoto Miyara1, Florence Tubach, Valérie Pourcher, Capucine Morélot-Panzini, Julie Pernet, Julien Haroche, Said Lebbah, Elise Morawiec, Guy Gorochov, Eric Caumes, Pierre Hausfater, Alain Combes, Thomas Similowski, Zahir Amoura Low rate of daily smokers in patients with symptomatic COVID-19 Preprint

2] Kerstin Frie and Kome Gbinigie Chloroquine and hydroxychloroquine: Current evidence for their effectiveness in treating COVID-19 Oxford COVID-19 Evidence Service Team Centre for Evidence-Based Medicine, Nuffield Department of Primary Care Health Sciences University of Oxford

3] A. J. Scheen Metformin and COVID-19: From cellular mechanisms to reduced mortality: Diabetes Metab. 2020 Aug. 1

4] Monica Jaramillo; Famotidine linked to improved outcomes in COVID-19 Front Immunol. 2020; 11: 1712. Aug. 17, 2020 Published online 2020 Jul. 10. doi: 10.3389/fimmu.2020.01712

5] Inga Wessels, Benjamin Rolles, and Lothar Rink: The Potential Impact of Zinc Supplementation on COVID-19 Pathogenesis

6] Kai, H., Kai, M. Interactions of coronaviruses with ACE2, angiotensin II, and RAS inhibitors—lessons from available evidence and insights into COVID-19. Hypertens Res 43, 648-654 (2020).

7] Romer Gonzalez-Villalobos 1, R Bryan Klassen, Patricia L Allen, L G Navar, Timothy G Hammond Megalin binds and internalizes angiotensin II Am J Physiol Renal Physiol. 2005 February; 288(2): F420-7.

8] Treatment with high dose vitamin B12 been shown to be safe for more than 50 years: Stichting Takort

I claim:

1. Treatment agents for Covid 19 comprising compounds with isoelectric points that match megalin receptor sites block the binding of Covid 12 virus on cellular surfaces and therefore prevent the entrance of Covid 19 virus into the cell.

2. Compound of claim 1 wherein said compound is cobalamin.

3. Cobalamin of claim 2 is administered within at least in one milligram per cubic centimeter of liquid.

4. Cobalamin of claim administered as an inhalation mist.

5. Cobalamin of claim 2 is administrated intravenously.

6. Cobalamin of claim 2 wherein said cobalamin plus one of more of compounds from the following: thiamin, amprolium or zinc.

7. Compounds of claim 6 wherein said compounds are administered vial oral route.
